(19) United States
(12) Patent Application Publication
(10) Pub. No.: US 2024/0218039 A1
(43) Pub. Date: Jul. 4, 2024

(54) MICROORGANISM EXPRESSING VASOACTIVE INTESTINAL PEPTIDE, AND USE THEREOF

(71) Applicant: LIVEOME INC., Suwon-si, Gyeonggi-do (KR)

(72) Inventors: Ji Yoon SONG, Seongnam-si, Gyeonggi-do (KR); Hyo Jeong CHOI, Seongnam-si, Gyeonggi-do (KR); Hyeon Jin NOH, Seoul (KR); Young Ha PARK, Suwon-si, Gyeonggi-do (KR); Yi Reh JUNG, Uiwang-si, Gyeonggi-do (KR)

(21) Appl. No.: 17/996,436

(22) PCT Filed: Apr. 9, 2021
(86) PCT No.: PCT/KR2021/004479

§ 371 Date: Oct. 18, 2022

(30) Foreign Application Priority Data
Apr. 20, 2020 (KR) 10-2020-0047660

(51) Int. Cl. C07K 14/575; A61K 35/747; A61P 1/00; A61P 29/00

(52) U.S. Cl. CPC C07K 14/57563; A61K 35/747; A61P 1/00; A61P 29/00

(57) ABSTRACT

Provided are a recombinant microorganism expressing a vasoactive intestinal peptide (VIP) gene, and a composition including the microorganism for preventing or treating a disease causing damage to the gastrointestinal tract.

### TECHNICAL FIELD

The present disclosure relates to a microorganism expressing vasoactive intestinal peptide (VIP), and a composition including the microorganism, for preventing or treating a disease causing damage to the gastrointestinal tract.

### BACKGROUND ART

VIP is a vasoactive intestinal peptide hormone. VIP is a peptide with 28 amino acid residues belonging to the glucagon/secretin superfamily, and is a ligand of class II G protein-coupled receptors.

Korean Patent Publication No. 2014-0000620 discloses Lactobacillus that increases VIP levels in the enteric nervous system and its use for treating irritable bowel syndrome (IBD). The microorganism is a strain screened from nature and is not a recombinant microorganism.

Chinese Patent Publication No. 108753670 discloses a recombinant microorganism Lactococcus lactis NZ9000 that expresses VIP and has abundant selenium nanoparticles (SeNP) in the cells. It is described that when this recombinant microorganism is used as a selenium additive, the issues of selenium's strong toxicity, low bioavailability, high potential to cause environmental pollution, and high production costs may be solved because selenium is present in the cells in the form of nanoparticles, and at the same time, an issue of residual antibiotics may be solved because the microorganism secretes VIP, an antibacterial peptide. In addition, the pNZ8148 plasmid of the NICER Lactococcus lactis expression system was used for VIP secretion expression.

However, a recombinant microorganism of the genus Lactobacillus expressing exogenous VIP, recombinant lactic acid bacteria expressing exogenous VIP under a constitutive promoter, and the use of such a microorganism in humans to prevent or treat a disease causing damage to the gastrointestinal tract, such as IBD, are not known.

### DESCRIPTION OF EMBODIMENTS

### Technical Problem

A purpose of the present disclosure is to provide a recombinant microorganism of the genus Lactobacillus expressing exogenous VIP.

Another purpose is to provide recombinant lactic acid bacteria expressing exogenous VIP under a constitutive promoter.

Still another purpose is to provide a composition including the microorganism for preventing or treating a disease causing damage to the gastrointestinal tract in humans.

### Solution to Problem

An aspect of the present disclosure provides a recombinant microorganism of the genus Lactobacillus, in which a promoter and an exogenous gene are introduced, and a protease is inactivated, wherein the exogenous gene is operably linked to the promoter and encodes vasoactive intestinal peptide (VIP).

Another aspect of the present disclosure provides a recombinant microorganism which is a lactic acid bacterium, in which a constitutive promoter and an exogenous gene are introduced, and a protease is inactivated, wherein the exogenous gene is operably linked to the promoter and encodes vasoactive intestinal peptide (VIP).

Still another aspect of the present disclosure provides a composition for preventing or treating a disease causing damage to the gastrointestinal tract in humans, including a recombinant microorganism, which is a lactic acid bacterium, in which a promoter and an exogenous gene are introduced, and a protease is inactivated, wherein the exogenous gene is operably linked to the promoter and encodes vasoactive intestinal peptide (VIP).

Hereinafter, the disclosure will be described in detail.

The term “VIP” or “VIP protein” or “VIP polypeptide” used herein refers to a biologically active polypeptide having one or more of the biological activities described herein.

In humans, the q25 region of chromosome 6 in the human genome encodes a member of the secretin family that is 170 amino acids in length. This member of 170 amino acids in length is post-translationally truncated to form vasoactive intestinal peptide (VIP). The active form of the VIP polypeptide functions to lower blood pressure, increase vasodilation of blood vessel walls, relax smooth muscle in the tissues of the respiratory system and gastrointestinal tract, decrease the immune response through the promotion of Th2 as well as reduction of the Th1 response, regulate the innate and acquired immune response, or promote the secretion of electrolytes in the intestine. It has also been shown that VIP is active in the central nervous system as a neurotransmitter and in communication with lymphocytes. The bioactivity of VIP is transmitted through three known receptors: VIP₁R, VIP₂R, and PAC₁R. These receptors are known to cause CAMP enrichment as well as production of intracellular calcium. Like VIP, the affinity of the receptors for secretins depends on the subtype and the amino acid sequence of the ligand. Human natural VIP has a short half-life, of about 2 minutes in the bloodstream.

VIP referred to herein includes a variant that mimics the function of natural VIP. The variant includes one having an increased half-life than natural VIP and equivalent or greater bioactivity than natural VIP.

As used herein, VIP includes, but is not limited to, human VIP, recombinant human VIP, rat VIP, and/or recombinant mouse VIP. VIP may be one having 50% or more, 60% or more, 70% or more, 85% or more, 90% or more, 95% or more, 98% or more, 99% or more, or 100% sequence identity with the amino acid sequence of SEQ ID NO: 1. VIP may each have the amino acid sequence of SEQ ID NO: 1, 2, 3, 4, 5, 6, 7, 8, or 9. VIP of SEQ ID NO: 1 is human-derived natural VIP. VIP having the amino acid sequence of SEQ ID NO: 2, 3, 4, 5, 6, 7, 8, and 9 are respectively, VIP1, VIP2, VIP3, VIP4, VIP5, VIP6, VIP7, and VIP8, VIP variants of human-derived natural VIP.

The VIP polypeptides used herein may be isolated from a variety of sources, such as human tissue or other sources, or prepared by recombinant or synthetic methods.

The term “VIP polypeptide” also includes variants of VIP polypeptides. Herein, VIP may be modified in such a way that a chimeric molecule is formed including VIP fused to another heterologous polypeptide or amino acid sequence.

As used herein, “protease” is a generic term for enzymes that degrade proteins, and may include any type known in the art without particular limitation.

In the present specification, “auxotroph” refers to a strain that cannot grow in a synthetic medium consisting only of inorganic salts and a carbon source and grows only when one or more nutrients are supplemented, and any known kind in the art may be included without particular limitation.

The microorganism according to an aspect of the present disclosure is a recombinant microorganism, which is of the genus Lactobacillus, in which a constitutive promoter and an exogenous gene are introduced and a protease is inactivated, wherein the exogenous gene is operably linked to the promoter and encodes vasoactive intestinal peptide (VIP).

The microorganism according to another aspect of the present disclosure is a recombinant microorganism, which is a lactic acid bacterium, in which a promoter and an exogenous gene are introduced and a protease is inactivated, wherein the exogenous gene is operably linked to the promoter and encodes vasoactive intestinal peptide (VIP).

The lactic acid bacterium may belong to the genus of Lactobacillus, Lactococcus, Bifidobacteria, Streptococcus, Leuconostoc, Weissella, Pediococcus, or Enterococcus, in particular, to the genus of Lactobacillus or Lactococcus, and may be, for example, Lactobacillus paracasei, Lactobacillus brevis, Lactobacillus plantarum or Lactococcus lactis.

The microorganism of the genus Lactobacillus may be Lactobacillus paracasei, Lactobacillus brevis, or Lactobacillus plantarum. For example, the microorganism may be Lactobacillus plantarum LMT1-9 (KCTC 13421BP), Lactobacillus paracasei LMT1-21 (KCTC 13422BP), or Lactobacillus brevis LMT1-46 (KCTC 13423BP).

The promoter may be a constitutive promoter. The promoter may have transcription initiation efficiency in lactic acid bacteria equal to or greater than that of a promoter in the art, for example, the P11 promoter. The promoter may also be an inducible promoter. The inducible promoter may be, for example, a Nisin inducible promoter. The promoter may be PR4 derived from Lactobacillus paracasei including the polynucleotide of SEQ ID NO: 10. PR4 may consist of the nucleotide sequence of SEQ ID NO: 11.

The microorganism according to the present disclosure is characterized by having an exogenous gene encoding VIP introduced. The introduction may be by transformation, transduction, transfection or electroporation. The introduced exogenous gene may be integrated into the genome of the host cell or may exist without being integrated. For example, the exogenous gene may be integrated into the genome of the host cell.

In addition, the microorganism according to the present disclosure is characterized by having a protease inactivated. In an embodiment, the gene encoding a protease may be deleted. “Deletion” includes deletion of at least a portion, for example, all or part of a gene.

According to an embodiment, the gene encoding a protease is replaced with an exogenous gene, which is operably linked to a promoter and encodes vasoactive intestinal peptide (VIP), whereby the introduction of the exogenous gene and deletion of the protease gene may be implemented simultaneously. “Replacement” includes replacement of at least a portion, for example, all or part of a gene.

In the present specification, “protease” may include any type known in the art without particular limitation. For example, the protease may be at least one selected from the group consisting of high temperature requirement A (HtrA), Aminopeptidase N (PepN), Caseinolytic protease P (ClpP) and Lon protease (Lon), and may be, for example, HtrA and/or PepN, or for example, HtrA. The htrA gene may be a L. brevis htrA gene, particularly, the gene of SEQ ID NO: 14. The pepN gene may be a L. brevis pepN gene, particularly, the gene of SEQ ID NO: 15. The clpP gene may be a L. brevis clpP gene, particularly, the gene of SEQ ID NO: 16. The Ion gene may be a L. brevis Ion gene, particularly, the gene of SEQ ID NO: 17. In these microorganisms, the yield or productivity of the VIP protein may be increased by preventing the exogenous VIP protein from being degraded because the protease is not expressed. According to an embodiment, when HtrA is not expressed, it is possible to significantly increase the productivity of the VIP protein.

The recombinant microorganism may further include a signal sequence operably linked between the promoter and the exogenous gene. When the signal sequence is linked in frame with the exogenous protein gene, the ability to secrete the exogenous protein out of the cell may be greater than that of a signal sequence in the art, for example, a USP45 signal sequence. The signal sequence may be one encoding SP4 (SEQ ID NO: 12) derived from Lactobacillus paracasei. For example, SP4 may have the nucleotide sequence of SEQ ID NO: 13. The microorganism may secrete VIP out of the cell.

In an embodiment, the recombinant microorganism may be an auxotroph. The auxotroph may be, for example, one in which is deleted at least one gene selected from the group consisting of riboflavin biosynthesis protein gene (ribB), thymidylate synthase gene (thyA) and glutamine-fructose-6-phosphate aminotransferase gene (glmS), for example, ribB and/or thyA, for example, ribB, but is not particularly limited thereto. ribB may be L. brevis ribB, in particular of SEQ ID NO: 18. thyA may be L. brevis thyA, particularly of SEQ ID NO: 19. glmS may be L. brevis glmS, in particular of SEQ ID NO: 20. In this case, environmental safety may be enhanced by controlling the growth and survival of the recombinant microorganisms. “Deletion” includes deletion of at least a portion, for example, all or part of a gene.

Another aspect of the present disclosure provides a composition including the above-mentioned microorganism, for use in preventing or treating a disease causing damage to the gastrointestinal tract.

The disease may be one that causes inflammation of the gastrointestinal tract. The disease may be at least one selected from the group consisting of inflammatory bowel disease (IBD) and colitis. The inflammatory bowel disease may be ulcerative colitis or Crohn's disease.

The composition may include a pharmaceutically acceptable or sitologically acceptable carrier, excipient or diluent. The pharmaceutically acceptable or sitologically acceptable carrier may be any standard pharmaceutically acceptable or sitologically acceptable carrier such as a phosphate buffered saline solution, 5% aqueous solution of dextrose and an emulsion (for example, oil/water or water/oil emulsion). Non-limiting examples of excipients include adjuvants, binders, fillers, diluents, disintegrants, emulsifiers, wetting agents, lubricants, glidants, sweetening agents, flavoring and coloring agents. The preferred pharmaceutically or sitologically acceptable carrier depends on the intended mode of administration of the active agent. Typical modes of administration include enteral (for example, oral) administration. The composition may be in a unit dosage formulation. The composition may have an oral dosage formulation. The composition may be a food or a pharmaceutical composition. The composition may include dry matter of the microorganism. The composition may include a medium of the microorganism.

As used herein, “pharmaceutically acceptable or sitologically acceptable” refers to one substantially not causing adverse reactions when administered to a subject. The adverse reaction may be toxicity, allergy, or an immune response.

Another aspect provides a kit for use in preventing or treating a disease causing damage to the gastrointestinal tract, including the above recombinant microorganism and another drug for treating a disease damaging the gastrointestinal tract. The recombinant microorganism is as described above. The recombinant microorganism may be in the form of the composition described above. The kit may include instructions for using the recombinant microorganism and the other drug for treating the disease causing damage to the gastrointestinal tract to prevent or treat the disease causing damage to the gastrointestinal tract.

The other drug for treating a disease damaging the gastrointestinal tract includes any drug that treats a disease damaging the gastrointestinal tract, for example, inflammatory bowel disease. Drugs for treating a disease damaging the gastrointestinal tract may include the following drugs:

### (i) Steroidal Anti-Inflammatory Agents

Dexamethasone, hexestrol, methimazole, betamethasone, triamcinolone, triamcinolone acetonide, fluocinonide, fluocinolone acetonide, predonisolone, methylpredonisolone, cortisone acetate, hydrocortisone, fluorometholone, beclomethasone dipropionate, estriol, paramethasone acetate, fludrocortisone acetate, clobetasol propionate, diflorasone acetate, dexamethasone propionate, difluprednate, betamethasone dipropionate, budesonide, diflucortolone valerate, amcinonide, halcinonide, mometasone furoate, hydrocortisone butyrate propionate, flumetasone pivalate, clobetasone butyrate, dexametasona acetate, etc.

(ii) 5-aminosalicylic Acid

Sulfasalazine, mesalazine, olsalazine, balsalazide, etc.

(iii) Immunomodulators or Immunosuppressants

Methotrexate, cyclophosphamide, MX-68, atiprimod dihydrochloride, BMS-188667, CKD-461, rimexolone, cyclosporine, tacrolimus, gusperimus, azathiopurine, antilymphocyte serum, freeze-dried sulfonated normal immunoglobulin, erythropoietin, colony stimulating factor, interleukin, interferon, etc.

### (iv) JAK Inhibitors

Tofacitinib, ruxolitinib, etc.

### (v) TNF Inhibitors

Recombinant TNF-alpha-receptor IgG-Fc fusion protein (etanercept), infliximab, adalimumab, certolizumab pegol, golimumab, PASSTNF-α, soluble TNF-α receptor, TNF-α binding protein, anti-TNF-α antibody, CDP571, etc.

### (vi) Integrin Inhibitors

Natalizumab, vedolizumab, AJM300, TRK-170, E-6007, etc.

(vii) Interleukin-12/23 Inhibitors

Ustekinumab, briakinumab (anti-interleukin-12/23 antibody), etc.

(viii) Non-Steroidal Anti-Inflammatory Drugs (NSAIDs)

### (a) Classical NSAIDs

Alcofenac, aceclofenac, sulindac, tolmetin, etodolac, fenoprofen, thiaprofenic acid, meclofenamic acid, meloxicam, tenoxicam, lornoxicam, nabumeton, acetaminophen, phenacetin, ethenzamide, sulpyrine, antipyrine, migrenin, aspirin, mefenamic acid, flufenamic acid, diclofenac sodium, ketophenylbutazone, loxoprofen sodium, phenylbutazone, indomethacin, ibuprofen, ketoprofen, naproxen, oxaprozin, flurbiprofen, fenbufen, pranoprofen, floctafenine, piroxicam, tenoxicam, epirizole, tiaramide hydrochloride, zaltoprofen, gabexate mesylate, camostat mesylate, ulinastatin, colchicine, probenecid, sulfinpyrazone, bucolome, benzbromarone, allopurinol, sodium aurothiomalate, hyaluronate sodium, sodium salicylate, salicylic acid, atropine, scopolamine, levorphanol, oxymorphone, a salt thereof, etc.

### (b) Cyclooxygenase Inhibitors (COX-1 Selective Inhibitors, COX-2 Selective Inhibitors, Etc.)

Salicylic acid derivatives (for example, celecoxib, aspirin), etoricoxib, valdecoxib, diclofenac sodium, indomethacin, loxoprofen, etc.

### (c) Nitric Oxide-Releasing NSAID.

The description of the microorganism is also applicable to the composition.

Another aspect provides a method of preventing or treating a disease causing damage to the gastrointestinal tract, including administering to the subject the microorganism of an amount that is effective for preventing or treating the disease causing damage to the gastrointestinal tract.

The method may further include administering to the subject a TNF-alpha blocker of an amount effective to prevent or treat a disease causing damage to the gastrointestinal tract. The administration of the TNF-alpha blocker to the subject may be performed simultaneously, before, or after the administering of the microorganism to the subject. The TNF-alpha blocker may be administered via a route the same with or different from the route through which the microorganism is administered. For example, the TNF-alpha blocker may be administered orally or parenterally. The parenteral route may be an injection such as vascular injection, intraperitoneal injection, or subcutaneous injection. For example, the TNF-alpha blocker may be administered intraperitoneally, and the microorganism may be administered orally. The TNF-alpha blocker may be administered intraperitoneally after the microorganism is orally administered. The TNF-alpha blocker may be administered in an amount of 5 mg to 100 mg, 5 mg to 50 mg, 5 mg to 40 mg, 10 mg to 50 mg, or 20 mg to 50 mg per administration.

In the method, the disease may be one causing inflammation of the gastrointestinal tract. The disease may be at least one selected from the group consisting of inflammatory intestinal disease, autoimmune disease, damage of the gastrointestinal tract by radiation or graft versus host disease (GVHD), inflammatory bowel disease (IBD), and colitis including chronic colitis. The inflammatory bowel disease may be ulcerative colitis or Crohn's disease.

As used herein, the “administering” or “administration” refers to acts of giving the microorganism, a composition including the same, or a therapeutic treatment to a physiological system (an organism, in vivo, in vitro, or ex vivo cells, a tissue or an organ). Accordingly, in the method, the microorganism may be in a form of the composition described above. An acceptable route of administration to the human body may be oral, or mucosal (for example, intestinal mucosa, oral mucosa or buccal). The administration may be in combination with an administration of an additional therapeutic agent. The combined administration includes any of simultaneous and sequential administrations.

As used herein, the term “treatment” refers to prophylactic treatment or therapeutic treatment. In certain embodiments, “treatment” refers to administration of microorganisms or a composition to a subject for therapeutic or prophylactic purposes.

A “therapeutic” treatment is a treatment administered to a subject exhibiting pathological signs or symptoms to reduce or eliminate the signs or symptoms. Signs or symptoms may be biochemical, cellular, histological, functional or physical, subjective or objective.

A “prophylactic” treatment is a treatment administered to a subject that does not show signs of a disease or only shows early signs of a disease in order to reduce the risk of developing a pathology. The microorganism or composition described herein may be provided as a prophylactic treatment to reduce the likelihood of developing a pathology, or to minimize the severity of the pathology when it does occur.

As used herein, “therapeutically effective amount” refers to an amount sufficient to carry out the stated purpose. An effective amount may be determined empirically. An effective amount may be determined, for example, depending on the extent to which the microorganism secretes VIP out of the cell. For example, for a person having a body weight of 60 kg, an effective amount may be the number of the microorganisms capable of secreting 0.01 to 300 mg, or 0.5 to 100 mg of VIP per day. For example, an effective amount may be 1×10⁵ cfu to 1×10¹³ cfu, 1×10⁶ cfu to 1×10¹³ cfu, 1×10⁷ cfu to 1×10¹³ cfu, 1×10⁷ cfu to 1×10¹² cfu, 1×10⁷ cfu to 1×10¹¹ cfu, 1×10⁷ cfu to 1×10¹⁰ cfu, 1×10⁸ cfu to 1×10¹¹ cfu, 1×10⁸ cfu to 1×10¹⁰ cfu, 2.5×10⁸ cfu to 7.5×10⁹ cfu, 5.0×10⁸ cfu to 5.0×10⁹ cfu, 7.5×10⁸ cfu to 2.5×10⁹ cfu, or about 1×10⁹ cfu per administration.

In the method, the subject may be a mammal. The mammal may be a human, horse, pig, cow, dog, cat, monkey, chimpanzee, sheep, or goat.

The description of the microorganism and/or composition is also applicable to the method.

### Advantageous Effects of Disclosure

The recombinant microorganism according to the present disclosure not only is able to produce and secrete VIP with higher productivity, but also exhibits high environmental safety, thereby more effectively prevents or treats a disease causing damage to the gastrointestinal tract.

### BRIEF DESCRIPTION OF DRAWINGS

FIGS. 1A and 1B are schematics showing genetically engineered sites of recombinant microorganisms according to an example.

FIGS. 2A and 2B are figures confirming the generation of recombinant microorganisms with improved VIP productivity, according to an example.

FIGS. 3A and 3B are figures showing that recombinant microorganisms according to an example had improved VIP productivity.

FIGS. 4A and 4B are figures confirming the generation of recombinant microorganisms with improved environmental safety, according to an example.

FIGS. 5A and 5B are graphs showing recombinant microorganisms according to an example had improved environmental safety.

FIG. 6 is a graph showing that a recombinant microorganism according to an example exhibits a therapeutic effect on IBD in an animal model.

### BEST MODE

Hereinafter, the disclosure will be described in more detail through examples. However, these examples are for illustrative purposes only, and the scope of the present disclosure is not limited in any way by these examples.

### Example 1: Preparation of Recombinant Microorganism

A recombinant microorganism expressing exogenous VIP was prepared, according to the homologous recombination genetic engineering method commonly used for lactic acid bacteria (Zhang et al., D-Ala-D-Ala ligase as a broad host-range counterselection marker in vancomycin resistant lactic acid bacteria, J. Bacteriol., 2018), using Lactobacillus brevis LMT1-46 (KCTC 13423BP).

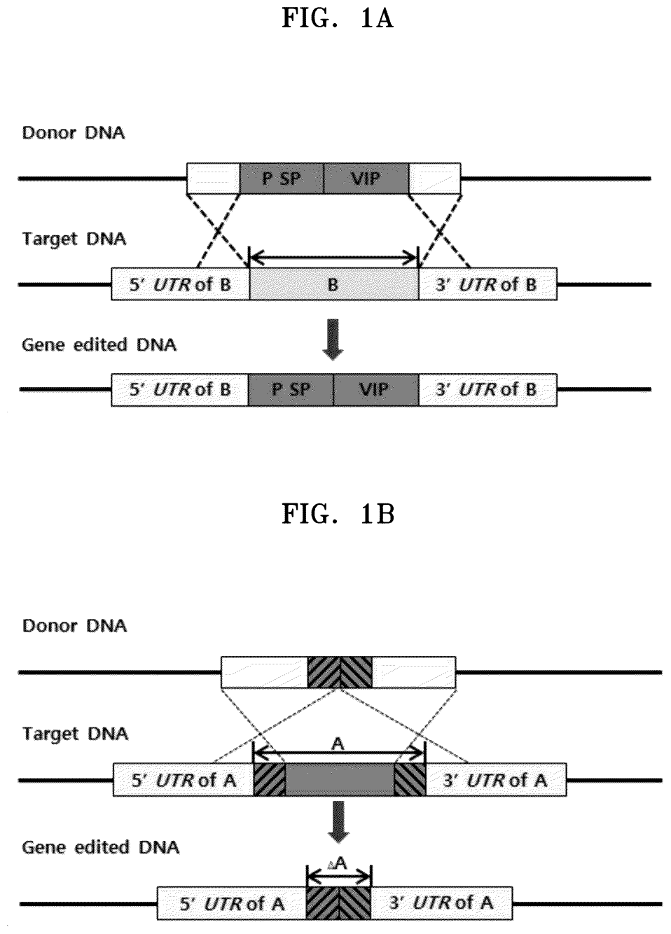

FIGS. 1A and 1B schematically show the genetically engineered site of the microorganism according to the example. FIG. 1A shows the process of replacing the protease gene (htrA or pepN) with the VIP gene to improve VIP productivity. A signal peptide that assists in secretion on 5′-side of the VIP gene and a tag for detection on 3′-side of the VIP gene were included in one cassette. The protease gene is L. brevis htrA (SEQ ID NO: 14) or L. brevis pepN (SEQ ID NO: 15). FIG. 1B shows a process of generating an auxotroph to promote environmental safety, by removing some regions of a target gene. The target gene is L. brevis ribB (SEQ ID NO: 18). Each abbreviation in FIGS. 1A and 1B has the following meaning: P: Promoter, SP: Signal peptide, UTR: Untranslated region, VIP: Vasoactive intestinal peptide), A: L. brevis ribB, B: L. brevis htrA or L. brevis pepN.

### Example 2: Confirmation of Generation of Recombinant Microorganism

PCR was proceeded to confirm that the recombinant microorganism was produced as intended. Lactic acid bacteria colonies stationary cultured at 37° C. for 16 hours on a De Man, Rogosa and Sharpe agar (MRS) plate were used. A single colony was picked and suspended in a tube containing 100 μl of distilled water. After bead-beating, the suspension was heated at 98° C. for 10 minutes to use as a DNA template. 1 μl of DNA template, 1 μl of forward primer, 1 μl of reverse primer, and 25 μl of DNA polymerase mix were mixed to prepare a PCR mixture. PCR was performed under the conditions of 40 cycles of amplification of (a) denaturation, 10 seconds at 98° C.; (b) annealing, 15 seconds at 55° C.; and (c) extension, 10 seconds at 72° C. per cycle, and a final extension of 2 minutes at 72° C. The primers used were designed to target regions outside the open reading frame (ORF) of the gene region to be engineered, and the sequences are described in Table 1 below.

TABLE 1

| | | SEQ |
|---|---|---|
| | | ID |
| Name | Sequence | NO |
| 1-46 pepN icF | AGC AAC CTT | 21 |
| | TGA CCT AGC | |
| 1-46 pepN icR | AAT TCC ATA | 22 |
| | TCA CCA CCC | |
| | AC | |
| pepN orf cF | CCC GAT GGC | 23 |
| | CTT ACA AC | |
| pepN orf cR | GGA ACG GCT | 24 |
| | GTC CAT TAG | |
| 1-46 htrA | CTA AAT GAG | 25 |
| insert cF | GAG GGT TCG | |
| | CG | |
| 1-46 htrA | AAG CTG GCG | 26 |
| insert cR | CTT TCA TTC | |
| | C | |
| 1-46 htrA | ACC TCT AAC | 27 |
| ORF cF | GTC AAC GTC | |
| 1-46 htrA | AGT TCC CAG | 28 |
| ORF cR | GGT TAA TCG | |

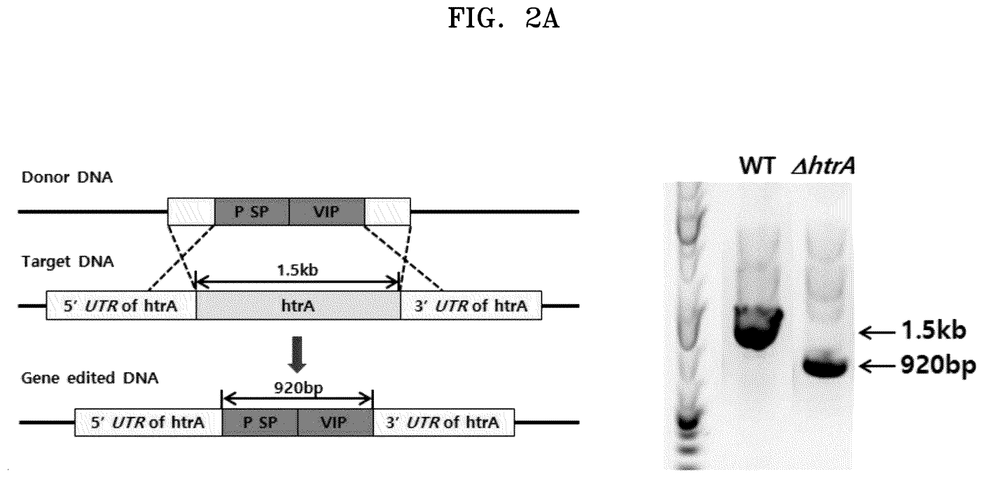

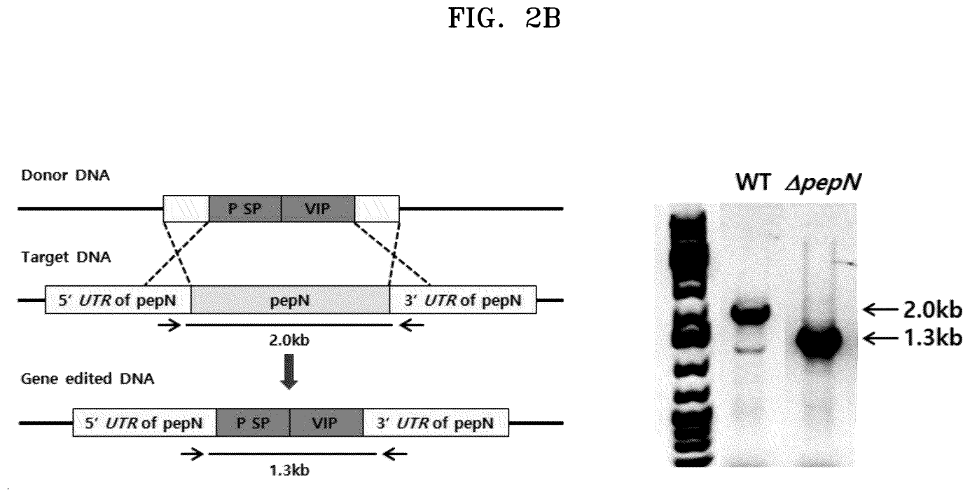

The results are each shown in FIGS. 2A and 2B. More specifically, FIGS. 2A and 2B are schematics (left) showing that the protease genes htrA (FIG. 2A) and pepN (FIG. 2B) are each replaced with a VIP expression cassette, and gel images (right) showing that the band sizes were changed as intended due to generation of recombinant microorganisms. As shown in FIGS. 2A and 2B, it was confirmed that recombinant microorganisms were generated in which the protease genes htrA and pepN were each replaced with a VIP expression cassette. In case of htrA, the band size of the PCR product was 1.5 kb in the wild-type, and when the htrA region was replaced with a VIP expression cassette, the band size was 920 bp. In case of pepN, the band size of the PCR product was 2 kb in the wild-type, and when the pepN region was replaced with a VIP expression cassette, the band size was 1.3 kb. In the schematics of FIGS. 2A and 2B, each abbreviation is as defined in Example 1, and WT in the gel image refers to the wild-type.

### Example 3: Evaluation of VIP Productivity of Recombinant Microorganism

In order to identify whether the VIP productivity of the recombinant microorganism was improved, an evaluation was performed to identify the VIP expression level of the strains prepared in Example 1. The stock of the prepared strain was streaked on an MRS plate and stationary cultured for 3 days at 37° C. Here, a single colony was picked and cultured with shaking at 37° C. in a MRS liquid medium for 24 hours. The cultured medium was inoculated into 25 ml of MRS so that OD₆₀₀ became 0.1, and then cultured with shaking at 37° C. for 16 hours. 10 ml of the strain culture medium was extracted and centrifuged at 4,000 rpm for 10 minutes, and only the supernatant was extracted, 1 ml of tricholoacetic acid (TCA) was added, and treated at 4° C. for 30 minutes or more. This was centrifuged at 10,000 rpm for 10 minutes, the supernatant was removed and the pellet was suspended using 1 ml of acetone stored at 4° C. After centrifugation at 13,000 rpm for 10 minutes, the supernatant was removed, and 0.5 ml of acetone was added to suspend the pellet. The suspension was again centrifuged at 13,000 rpm for 10 minutes, and dried at 60° C. for 5 minutes. A sample buffer (reducing buffer) was added and the pellet was mixed well and treated at 98° C. for 10 minutes. Western blotting was performed using the obtained solution. Based on the results of the western blotting, the intensities of the bands measured according to regions of the same size were quantified by using a program called ImageJ.

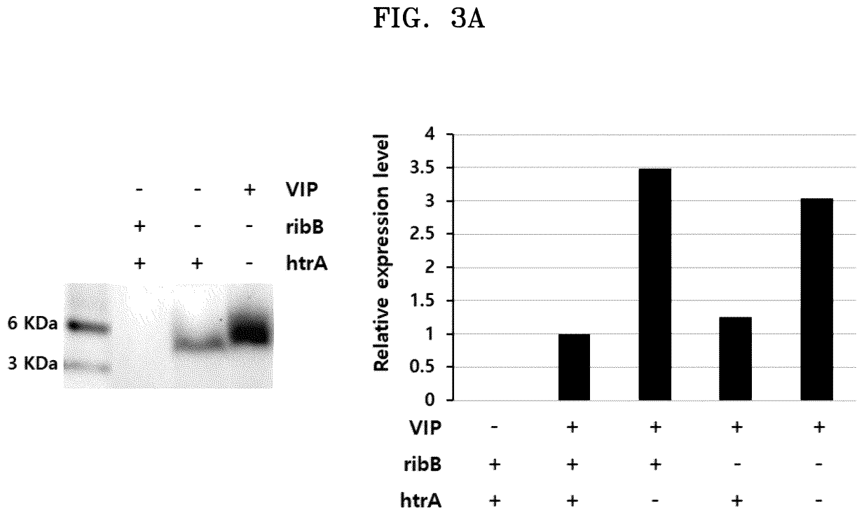

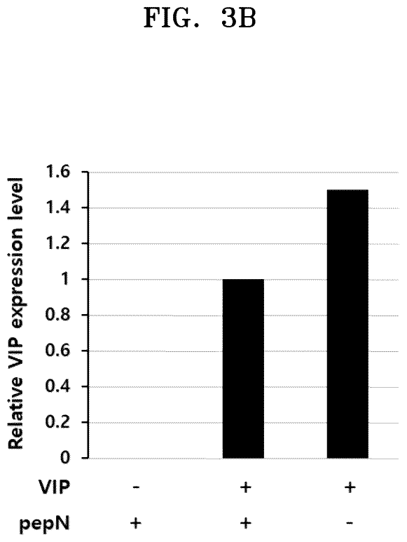

The results are each shown in FIGS. 3A and 3B. More specifically, FIG. 3A is a gel image (left) showing the generated recombinant strain in which htrA and/or ribB is deleted, and VIP expressed therefrom, and a graph (right) quantifying expression level of VIP. FIG. 3B is a graph showing expression level of VIP expressed from a recombinant strain in which pepN is deleted. As may be identified from FIGS. 3A and 3B, when the protease gene was deleted from L. brevis, the expression level of VIP, an effective substance, was increased. In particular, when htrA was deleted, the expression level of the target substance, VIP, was increased more than 3 times compared to those when other genes were deleted.

### Example 4: Confirmation of Generation of Auxotrophs

PCR was performed to confirm that the auxotroph was generated as intended. Lactic acid bacteria colonies, which were stationary cultured at 37° C. for 16 hours on a De Man, Rogosa and Sharpe agar (MRS) plate, were used. A single colony was picked and suspended in a tube containing 100 μl of distilled water. After bead-beating, the suspension was heated at 98° C. for 10 minutes to use as a DNA template. 1 μl of DNA template, 1 μl of forward primer, 1 μl of reverse primer, and 25 μl of DNA polymerase mix were mixed to prepare a PCR mixture. PCR was performed under the conditions of 40 cycles of amplification of (a) denaturation, 10 seconds at 98° C.; (b) annealing, 15 seconds at 55° C.; and (c) extension, 10 seconds at 72° C. per a cycle, and a final extension of 2 minutes at 72° C. The primers used were designed to target regions inside and outside the ORF of the gene region to be engineered, and the sequences are described in Table 2 below.

TABLE 2

| | | SEQ ID |
|---|---|---|
| Name | Sequence | NO |
| 1-46 ribB | TAA CCG CAG | 29 |
| orf cF | TGA CTG AC | |
| 1-46 ribB | AGC TGA TAC | 30 |
| orf cR | ATC AAA GGT | |
| | C | |
| 1-46 ribB icF | AGC ATT GTG | 31 |
| | TTA TCA GC | |
| 1-46 ribB icR | GCA GCA TTG | 32 |
| | GTA GCA AC | |
| LbthyA-UP-cF3 | GTG TGG CAA | 33 |
| | GGT GGC AAA | |
| | GCC A | |
| LbthyA-DN-cR1 | CCG ATC TAC | 34 |
| | AGG CCC AAC | |
| | TCG ATG A | |

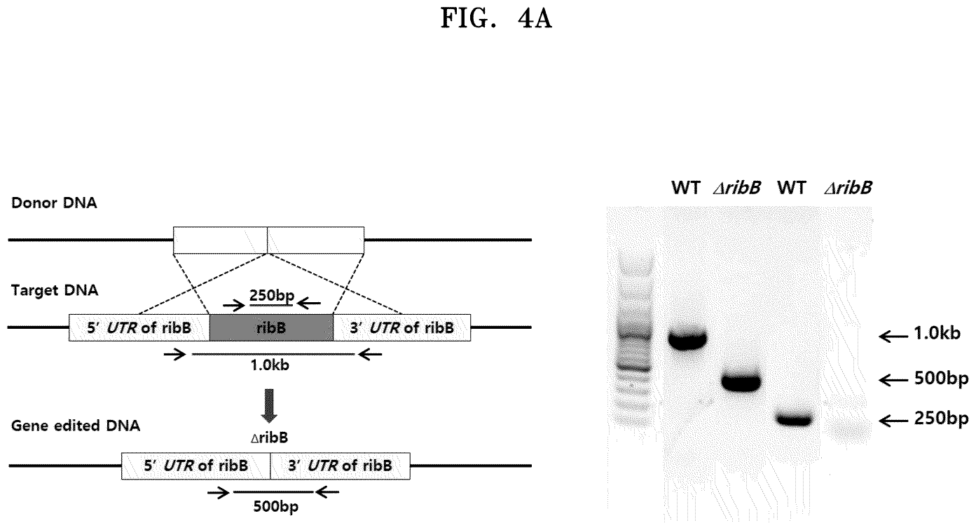

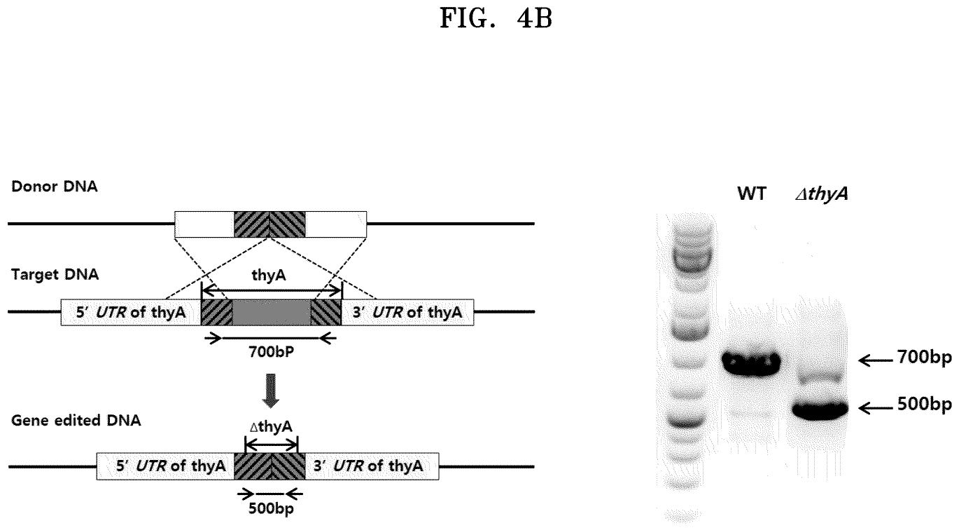

The results are each shown in FIGS. 4A and 4B. More specifically, FIGS. 4A and 4B are schematics (left) of recombinant microorganisms in which rib and thyA are deleted, respectively, and gel images (right) showing that the band sizes were changed as intended due to the generation of the recombinant microorganisms. In FIGS. 4A and 4B, each abbreviation is as defined in Example 1, and WT in the gel image refers to the wild-type. As shown in FIGS. 4A and 4B, it was confirmed that recombinant microorganisms were generated in which ribB and thyA were deleted, respectively. In case of ribB, the band size of the PCR product was 1.0 kb in the wild-type, and when the ribB region was deleted, the band size was 500 bp. When PCR was performed using a primer that binds to the inside of the ribB region, the wild-type was found to be 250 bp, and no band was observed in the strain in which ribB was deleted. In case of thyA, the band size of the PCR product was 700 bp in the wild-type, and when a part of the thyA region was deleted, the band size was 500 bp.

### Example 5: Evaluation of Environmental Safety of Auxotroph

A growth test was performed to determine the growth pattern of the strain prepared in Example 1. The stock of the prepared strain was streaked on an MRS plate and stationary cultured for 3 days at 37° C. From the plate, a single colony was picked and cultured with shaking (230 rpm) in an MRS liquid medium at 37° C. for 24 hours. 1 ml of the culture medium was extracted and centrifuged at 13,000 rpm for 1 minute. After suspending the cell pellet in 1 ml of 1x PBS solution, the cell was inoculated into 25 ml of a minimum medium for lactic acid bacteria which could limit the growth of lactic acid bacteria called semi-defined media (SDM), so that the measured OD₆₀₀ became 0.0025. It was cultured for 40 hours under a shaking culture (230 rpm) condition at 37° C., and the measured OD₆₀₀ values at 15, 17, 20, 22, 24, and 40 hours after inoculation are shown as a graph.

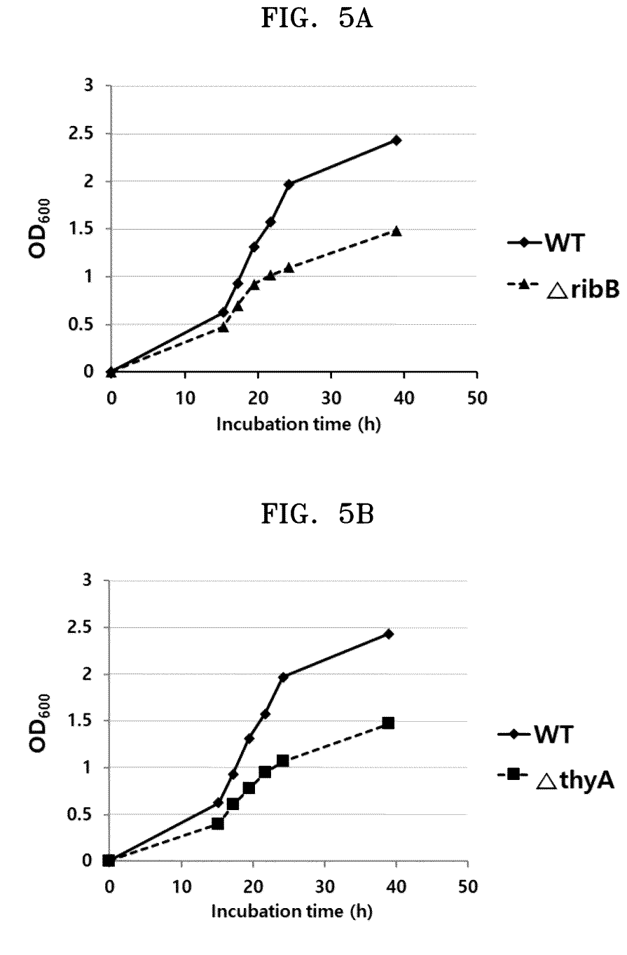

The results are each shown in FIGS. 5A and 5B. More specifically, FIGS. 5A and 5B are graphs showing the growth patterns of ribB-deleted recombinant lactic acid bacteria (FIG. 5A) and thyA-deleted recombinant lactic acid bacteria (FIG. 5B). In FIGS. 5A and 5B, WT means wild-type. It was confirmed from FIGS. 5A and 5B that the growth of the auxotrophic lactic acid bacteria has been inhibited in nutrient-deficient medium conditions.

### Example 6: In Vivo Efficacy: IBD Animal Model Experiment

In order to evaluate therapeutic efficacy of VIP protein derived from the transformed strain, the transformed strain was orally administered to mouse intestinal inflammation models induced by dextran sulfate sodium (DSS), and then the survival rate or disease activity index (DAI) score were identified. After primarily culturing in 5 ml of MRS medium for one day, the cultured strain was inoculated into 50 ml of MRS medium so that OD₆₀₀ became 0.1. When the OD₆₀₀ of the culture medium reached 4 to 5 after 16 to 18 hours, the strain was recovered according to the total number of mice to be administered and the number of feedings. The culture supernatant was removed by centrifugation at 4,000 rpm for 10 minutes, and the recovered strain was suspended in 1×PBS so that it could be administered at 1×109 cfu per mouse per administration. The administration of the strain was performed once a day, for a total of 16 days.

The treatment efficacy experiment was started by setting the first day of administrating the strain to Day-9, and from Day 0 to Day 5, 2% DSS was in drinking water in all groups except for the PBS group, and euthanasia was performed on Day 8. After DSS treatment, the DAI score was calculated by checking the weight loss, bristled hair, the degree of movement of the animals and the presence of diarrhea every two days.

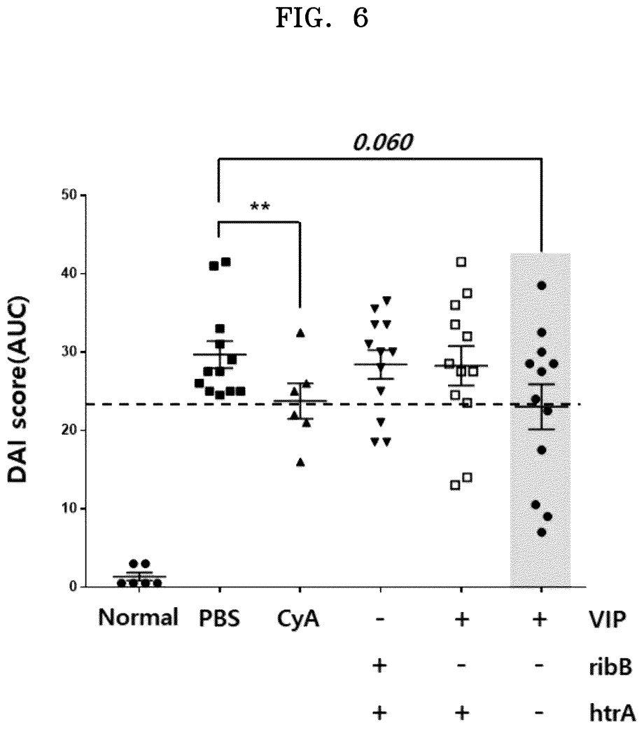

The results are shown in FIG. 6. As shown in FIG. 6, it was confirmed that the strain having improved productivity and improved environmental safety exhibited superior efficacy compared to the control groups in the IBD animal model experiment.

### ACCESSION NUMBER

Depository institute: Korea Research Institute of Bioscience and Biotechnology
Accession number: KCTC13421BP
Deposition date: 2017 Dec. 12
Depository institute: Korea Research Institute of Bioscience and Biotechnology
Accession number: KCTC13422BP
Deposition date: 2017 Dec. 12
Depository institute: Korea Research Institute of Bioscience and Biotechnology
Accession number: KCTC13423BP
Deposition date: 2017 Dec. 12

1. A recombinant microorganism of the genus Lactobacillus, in which a promoter and an exogenous gene are introduced and a protease is inactivated, wherein the exogenous gene is operably linked to the promoter and encodes vasoactive intestinal peptide (VIP).

2. The microorganism of claim 1, wherein the microorganism is Lactobacillus paracasei, Lactobacillus brevis, or Lactobacillus plantarum.

3. A recombinant microorganism, which is a lactic acid bacterium, in which a constitutive promoter and an exogenous gene are introduced and a protease is inactivated, wherein the exogenous gene is operably linked to the promoter and encodes vasoactive intestinal peptide (VIP).

4. The microorganism of claim 3, wherein the lactic acid bacterium is of the genus Lactobacillus or the genus Lactococcus.

5. The microorganism of claim 4, wherein the lactic acid bacterium is Lactobacillus paracasei, Lactobacillus brevis, Lactobacillus plantarum, or Lactococcus lactis.

6. The microorganism of claim 1, further comprising a signal sequence operably linked between the promoter and the exogenous gene.

7. The microorganism of claim 1, wherein a gene encoding a protease is deleted.

8. The microorganism of claim 7, wherein the gene encoding a protease is replaced with an exogenous gene operably linked to the promoter and encoding vasoactive intestinal peptide (VIP).

9. The microorganism of claim 1, wherein the protease is at least one selected from the group consisting of HtrA, PepN, ClpP, and Lon.

10. The microorganism of claim 1, wherein the microorganism is an auxotroph.

11. The microorganism of claim 10, wherein at least one gene selected from the group consisting of ribB, thy A, and glmS is deleted.

12. A composition for preventing or treating a disease causing damage to the gastrointestinal tract in humans, comprising a recombinant microorganism, which is a lactic acid bacterium, in which a promoter and an exogenous gene are introduced and a protease is inactivated, wherein the exogenous gene is operably linked to the promoter and encodes vasoactive intestinal peptide (VIP).

13. The composition of claim 12, wherein the disease is one causing inflammation of the gastrointestinal tract.

14. The composition of claim 13, wherein the disease is at least one selected from the group consisting of inflammatory bowel disease (IBD) and colitis.

15. The composition of claim 14, wherein the inflammatory bowel disease is ulcerative colitis or Crohn's disease.


SEQUENCE LISTING (34 sequences)

SEQ ID NO: 1 - protein, 28 aa, Homo sapiens
HSDAVFTDNYTRLRKQMAVKKYLNSILN

SEQ ID NO: 2 - protein, 28 aa, Artificial Sequence
  VIP variant 1
HSDAVFTDNYTRLRRQLAVRRYLNSILN

SEQ ID NO: 3 - protein, 30 aa, Artificial Sequence
  VIP variant 2
HSDAVFTANYTRLRRQLAVRRYLAAIGLRR

SEQ ID NO: 4 - protein, 25 aa, Artificial Sequence
  VIP variant 3
FTANYTRLRRQLAVRRYLAAILGRR

SEQ ID NO: 5 - protein, 28 aa, Artificial Sequence
  VIP variant 4
HSDGIFTDSYSRYRKQMAVKKYLAAVLN

SEQ ID NO: 6 - protein, 28 aa, Artificial Sequence
  VIP variant 5
HSDAVFTDNYARLRKQMAVKKALNSILA

SEQ ID NO: 7 - protein, 31 aa, Artificial Sequence
  VIP variant 6
HSDAVFTDNYTRLRKQVAAKKYLQSIKNKRY

SEQ ID NO: 8 - protein, 31 aa, Artificial Sequence
  VIP variant 7
HSDAVFTDNYTRLRRQLAVRRYLNSILNGRR

SEQ ID NO: 9 - protein, 23 aa, Artificial Sequence
  VIP variant 8
HSDAVFTDNYTRLRRQLAVRRYL

SEQ ID NO: 10 - DNA, 31 bp, Lactobacillus paracasei
tcgtcacggcgctgcttttttcatacaaaat

SEQ ID NO: 11 - DNA, 500 bp, Lactobacillus paracasei
tccacaatcaaggtgcttggctttttcgatcgcgaggtcaccatgtacatcagtcgtgagagcattgtgttgacagtgatcggcatcgtgttcggctatctgctcggcaatttgctgacagcctacattttgtatcaagccgaaactgaggccgtggtttttccactcacgatcagcattgtcggctacctcacggccacgttactcatgttggccttcaccggcgtcgtcacctggctcacgcatcgtcgactccaacgggtggacatggtcgaagccctgaaatcaaacgaataacctacaattttgtcaggcagcgtcgtcacggcgctgcttttttcatacaaaattcatcaaaaattgggattaaaaacgttcatgatcgcaattttgaagcgcaaatgaagattgagaccaactcctaacagtcctgtaacgctgacgtaacattgacacagtaaagtagcctttagttaatcaaattaagggtgaggtcaaaa

SEQ ID NO: 12 - protein, 31 aa, Lactobacillus paracasei
MKFNKVMITLVAAVTLAGSASAVTPVFADTS

SEQ ID NO: 13 - DNA, 93 bp, Lactobacillus paracasei
atgaaattcaataaagtcatgatcacgttggttgctgcagttaccttagcaggttctgctagcgccgtaacaccagttttcgctgatacaagc

SEQ ID NO: 14 - DNA, 1347 bp, Lactobacillus brevis
gtgatggataacaataataatcaatttaataaccaacaacatgatcataatgaagaaaccacaccaactccggaaccacggaaacggcggggtggcctgacactgacgaaagtggcagtgacggctgcggtcgctggactcttaggcggcggggttgcgtacggcggtatcaactactttaataatagtgggatgaatgatacggcggtgccagccgggagtaactcgaccgggaacaccaagacctctaacgtcaacgtcaatgtctcgagtcaatcaaccaaggcctttaataaggttaaaggggccatggtctcggtgattaatttacagaaggaaaatagtagtagcgggacgcttggccaattattcggctcaagctcgagctccaattcatcgaaatccaagtcagagctggaagaagcttccgaaggttctggggtgatttataagaaggacggtaacgtggcttacatcgtgacgaacaatcacgtggtttccggctcttcagccttacgagtcgtgacgagttccggtaaacaactgcaagcgaagttggttggtaaggattccgttaccgacttggccgtcttaaaggttaacggctcgtcattaaagaccgtggcgtcatttggaaactcagataatatcaaggttggggaaacggccttggcaattggctcaccactgggcagccaatacgcaacgtcattgacgcaagggatcatctccgccaagaagcggacgattgaaacgactaattcttcgggaacgcaaaccgggaatgcaacggttattcagacggatgcggcgattaaccctgggaactcagggggggccttgattaatgtcggtggccaagtggtcggcattacctcatcgaagatcgccagtgatgcggaaggcaccagcgttgaagggatgggctttgccattccatctaacgaagtggtcaatatcatcaatcaactggtcaagaacgggaaggtcgtgcggccagcgttagggattacctacgttgacttggcgaacgtttctagtgcgcaacagaaatcgatcctgaagttaccatctaatgtggaaaacggggtcgtggtgatgagtgcttcggcgggctcaccagcgaagaaggccggcttgagcaagtatgatgtgattacggcgctgggcggtcatcagatttcagaccaatccacgttgcgagatattttgtacaagtataagctgaacgataaggtctcgatcacttactatcacaatggtaagaagaagaccagcaccattaaattaacggaaacatcttctcaattaaagactagctcgacgacggcaacgtcagagagttag

SEQ ID NO: 15 - DNA, 2535 bp, Lactobacillus brevis
atgacagaatcaacgcacttgtatgacctgttccacccggcacactacgatatttatctcgatattaaccgcgaaacgaagcaaattagcgggaagacgacgattactggtgatgccttagccacgacgattggcattcaccagaagttcttaaagatccaacaggtaacggttgacggcaaagccgtgacggttacggatgatcctgcggccgaagccattcggattccggtcccacagacgggtaacgtgacgttagccattgattacacggcgccattaactgatagcatgatggggatttacccatcctactacgacgttaacggcgtacaaaagcaaatcattggaacccagtttgaaacgaccgctgcgcgccaagctttcccaagtatcgatgaaccggaggccaaagcaacctttgacctagcgattaagttcgatgaacagcctggtgaaacggttatcagtaacatgcctgagaccaaggtcgaagccggcgtgcactactttgacaccacggttaagatgtccacgtacctgattgcctttgcctttggtgatttgcaaagtaagatgacgactactaagagtggggttcaggttggggtgtttgctaccaaggcgcatcaagccaatgaattggactttgcgttagacattgccaagcgctcaattgaattttacgaagacttctatcagacaccttatccactaccacattcctggcagttagctttacctgacttttccgctggcgccatggaaaactggggattggttacttaccgggaagcttacctcgttttagaccccgacaatacgtcgtttgaaacgaagcaacgggttgccaccgtcattgctcatgaactggcccaccaatggtttggtgatttggttacgatgaagtggtgggatgatctttggttgaacgaaagttttgccaacatgatggaatacgttgctattgatgccatcgaacctgactggcacatttgggaagtcttccaaacaactgaagccccgatggccttacaacgggacgcaacggacggtgtacaatctgtccacgtgcaagtagaagatccagctgaaattgatgcgctctttgatagtgccattgtttacgccaagggtgcccggatgttagtcatggtacgggctttaattggcgatgatgctctgcgaacgggcctgaaggcttacttcgacgcccataagtttggtaatgcaactggggctgacttgtggtctgctttagggactgctgccaatatggatttgaagagcgtaatgaattcgtggctggaacaacctggttacccagtcttaacggctgcggtggtcgatggtcagttgacagtgacccagcaacaattctttattggcgatggcacagacgccaaacgtcaatggcaagtgccattgagcagtaactatgctgaagtcccagaattgttgacggcgccacaagtgacgttaggaacttatgcggatttacggcaggctaatggacagccgttccggctaaatgtcggcaataattcacatgcgattgtgaagtacgatgataccttactagcagatattttagatcacatcactgagttaacagcgattgatcaactccaattgctacaagatcaacgactactggctgaagggcggcagatttcttatgcggatgtcgtcgccttgttgccacgatttgcggctagtgaagctacggtggttaacgatgcgttgtatcaggtcgctggtaacttgaagaagttcgtcacgccagattctgatgccgaaaagcaactgcaacaattctttgatcggttaagtgcggcacaagttgaccggttggggtggacccctaaagctggcgaatctaatgatgaccagttaacccggccatatgtattaagcgcggccctctatgctaaaaatgcccaatcagtcgccgctgcgcatgaactgttcacagccaatcgcgacaatttagcaggcttgccagctgcaacgcgttactatgttttggcaaatgaagtcacgaacttcagtgacgcaacggtctttgatcagttgctgacagattaccggcaagcaacggatgccagctacaaggccgatctgtgtgccgcattgacgaccacaccagatgccaagttgattgcgaagttaatcgcagtctttgaaaacgcggccacggttaaaccacaggacttacgggcttggtaccgtggtgtcttggccaatgccaaggggcagcaggtggcttgggactggatccgtcaagattgggattggttggaagcaactgtgggtggtgatatggaattcaccacatttattacggtaactgcggcgatcttccacacacctgaacggttagctgagttcaaggaattctttgaaccaaagattccgacgcctggtttaggacgtgaaatcaagatggataccaaggttatcgcaagtcgcgtggccttagttgaagatgaaaaagccgcagtgaatgctgcggtggcacaagtcgtccaataa

SEQ ID NO: 16 - DNA, 597 bp, Lactobacillus brevis
atgaatttagtaccaacagttattgaacaatcatcccgtggcgaacgggcctatgatatttattcacgactattaaaagaccggattatcatgttatctggtccaattgaagatgacatggccaacgccatcatcgcgcagttactcttcttggatgcccaagactcaaccaaggacatttcactctacatcaactcacctggtggtgtcgtttcttccggcttggccatctatgataccatgaacttcatccaatctgacgtgcaaacgatcacgttagggatggcagcttccatggcaagtgtcttggcttcatctgggactaagggcaagcgttttgctttgcctcacgcccaagtgatgattcaccaaccatctggtggtgcccaaggacaacaaacggaaatcgaaattgctgctcgtgaaatcttgaagacccgtgaattgatcaacaagattttagcagagaattccggtcaaccgattgaacgcctcaatcaagatactgaacgagacaactacttgagtgctcaagaagcggttgattatggattaattgaccacatcatgaccaacagtagtgaacaaaagaagtaa

SEQ ID NO: 17 - DNA, 1050 bp, Lactobacillus brevis
gtgtggaatttaacgcgacggcagtggcggcagattctagtaacggtgatattagcacttgcagtgttgctattttttctgtggccattgcccaagtatattgagggtcccggtgaggcagacaatttgaaatcatttgtcacagttgcccaacatcccgataagcataagggaaaatatatgattacctcagttgcgttggcgcaagctcgtccagtttcctacctctatgccaaatttaatccctattacagcattgagagtgtagcgtcagtgacgggtggcgagagtaatgcgacctatgataaagttcagaacttttatatgaaaagcgccatcaatgagtcgatttataccgcatatcgggcggcccataaaacggtgaagcgtcattatttgggaatttacgttttgagtgttgatcgtcggtcaccctttgcccaagcgttaaaggtgggcgatacggtgacggaggtaaatggtcatcatttcaactctatgacggggtatcaacgatatatccagcgacaaaaagtgggacaacggacgaccattacgtatcagcacaatggtaaaacacaccacgtaactgccaaattgatgcggttgccaacgaaaaaagcgggtattgggattactttgacggataacgtcaaagttagtgctcggcctaaagtgcaggtgaatcccggcaacatcggtggtccgtcaggtggtttaatgtttagcttacagatttatacgcaagtgacggggcaggacttacgtcacgggcgaaagattgcaggaaccggaacggttgatggtgacggcaacgttggcgagattggtggtattgataagaaaattatcgctgctaagcgcgcaggggcgacggtcttctttgctccctacgtgaaaccgacaaaattgttactgaagtatgaagaacaacaccaaacgaattatcaattggctaaggcaacagcgaaaaagtatgcgccgaatatgaaagttgtgccagtaaaatcgtttgacgatgcagttcattacttaaggacacaccgttaa

SEQ ID NO: 18 - DNA, 606 bp, Lactobacillus brevis
atgttcacaggaataattacagatcaaggttacattcgacaaataaataaaacagccacgggagcacagctcgttattcaaacgacacgggctcatcagacacgacacgtcggggacagcatcgccgtgaacgggatctgcctcactttaaccgcagtgactgacgaagcatttaccgttgttgcaatggctgaaaccttgcagcgtacgacactgggaacttggcagactggtatgcaggtcaatctggaagctgctttacgagcagatcaaggattggatggacatatcgttcaggggcacgttgacaccacagcgaccttacgagcaagtcataccgaaggggccgcagtccggttgacctttgatgtatcagctgatcaagctccttacttggttgaaaagggtgctgtggccttggatggtgttagtttaacggtcacggcagttactcgtcagacggttcaggtagcgctgattccctatacgttggcacatacgagtcttggtcatctacagtgcggtgatcaggtgaatgtagagacagatattttggggaagtacgtggttcgtcagtaccagttaggggggatgactgatgactag

SEQ ID NO: 19 - DNA, 951 bp, Lactobacillus brevis
gatttgccggtgtccccggaattagcgaccaagtcccagtatagggcagtggaattacagtgagtagagtattagcttctatgagctgaatagtcctaaattggtgttgacaccgcgtcgtacaccacaggcgcgatcgacccaaaactaaacgagttgcactaatgacatttacacgcgcagtgggtcgcatacctgctttagcgggtggagattcgggtgaaggaatcgttagtcgctacagttgttacgcatcgaccgttataacttcccgtgtggatccttctatagtcgtgaagagaccatttcgactgtcgaattaaacggtagtaactgcattttaactttctcgcacaccgttccaccgtttcggtaactaccgtgtagtcggcctaaggtgcgactttgctagtaggcggctctcagacccacgcaggcgttgcagagttagtgtaaccggtcaactagttaccaaagtggaactactggaacggttcgggcggtgactgatggtatttgatctgggtttagtggtttcaccagtggttttcgaactaggcaatgctaagctagtgttttacggaaaaaaagacgaactataacgactaatttcagacgtaggtggttccttgccgctggtttcagccagtatagaccgggcactataagccctgatagttgcatcgcaactttccgggtgagcagggtttataccaacaccacaacgttatttttggcttataatcataggggtgcttccttggtgttctcaagcctaaattactctggtttcccgtggaaaaatcatcagttatcacccttcgggagaacgtttaatttggcgtaaaccatcggtttattcgattagcaaggccaagggcatgccaggcaaaaaagcactgggcagcagttctgtcagaatcgatttagatttatacggagaagattgtg

SEQ ID NO: 20 - DNA, 1818 bp, Lactobacillus brevis
atgtgtggaattgttggagtgacgggtaacgataacgccgttaaaattttagttaatgggctacaaaagttggaatatcgcggctatgattcagccggaatctatgttaatgaccaaaaaggccaggattatttggtaaaggccaagggccggatttccgaattaagtgctaagattacgccagaagttcacggatcaactgggattggtcatacgcgttgggcaacgcatggggtggttagcgtggataatgcgcacccacatttctccaacgacgaccgcttttacttggttcacaatggtgtgattgataacttccaagaacttaaggcgcaatatttaagtgatgtacctttccggagccaaacggatacagaagttgtcgtacagttgattgataagtttgcggtagaagatcacttagatgctaagcaggctttcttgaagacactaggcctgctgaagggttcctcatatgccttcttaatgatggatcgggaacaaccagacacattatttgtggctaagaacaagagccccttattaattggtgtcggcgatggcttcaacgttgtctgctcagatgccttggccatgttacgggaaacccacgatttcttggaattaatggatggtgaagttgtaacgatcacgccagataaagtggcaatcgaagacgctgatggtcagactgttgagcggaaacctttccacgttgacatgaacgctgatgaagcagacaagggcacttatccattctacatgttaaaagaagttgatgaacaacctaacgtgatgcgtaaattagcccaaacatatctgtcagaacacggcgaacctaacattgacaagcaattgctacaagcaatggaagccgctgatcgattgtatatcattggcgctgggacgagttaccacgctgggctgattggtaagcgcctctttgaaaacttggctcacattccaacggaagttcatgtgtcatcagaatttgcgtatgaacaaccgatgttatctgagaagccattctttattttcctgacccaatctggagaaacagctgatagtcgtgaagttttggttaacgtgaacgatgcgggttacccaagcttgacaatcaccaatgtgcaaaactccacgttatcacgtgaagccacttacaccttgttgttacatgccggtccagaaattgcggtggcttcaaccaaggcgtacacggcccaaattgctttggaagctatcttggccaaggctttaggtgaattaactggccagattattgcgcagaatttcaatattcggcaacagttgggactcgtcgcaactgggatgcaagccattgtggatgaaaaggataagctggaagaaatgtcttccaagtacttgttaaagactaaccgggcgttttacattggccgggggatcgaccacgccgtttccttagaagcagctttgaaactgaaagagatttcttatattcagtgtgaaggcttcgcgtctggtgaattaaagcatggcacgattgcattaattgaggctggaacaccagttattgggtttattacgcaggcgaagacggctggtttaacccggagtaacttacaagaaacgatggcccgtggtgcacaaacattgacgattgtccgggaaagtttagccgtagatggtgatgacttagtcttaccggatgtcgatgagatgttaatgccactcttaagtgttgtaccagcacaattgttggcctactacacgagtttaaataagggcttagatgttgataagccacggaacttggccaagagtgtgacagttgaataa

SEQ ID NO: 21 - DNA, 18 bp, Artificial Sequence
  primer
agcaacctttgacctagc

SEQ ID NO: 22 - DNA, 20 bp, Artificial Sequenc
  primer
aattccatatcaccacccac

SEQ ID NO: 23 - DNA, 17 bp, Artificial Sequence
  primer
cccgatggccttacaac

SEQ ID NO: 24 - DNA, 18 bp, Artificial Sequence
  primer
ggaacggctgtccattag

SEQ ID NO: 25 - DNA, 20 bp, Artificial Sequence
  primer
ctaaatgaggagggttcgcg

SEQ ID NO: 26 - DNA, 19 bp, Artificial Sequence
  primer
aagctggcgctttcattcc

SEQ ID NO: 27 - DNA, 18 bp, Artificial Sequence
  primer
acctctaacgtcaacgtc

SEQ ID NO: 28 - DNA, 18 bp, Artificial Sequence
  primer
agttcccagggttaatcg

SEQ ID NO: 29 - DNA, 17 bp, Artificial Sequence
  primer
taaccgcagtgactgac

SEQ ID NO: 30 - DNA, 19 bp, Artificial Sequence
  primer
agctgatacatcaaaggtc

SEQ ID NO: 31 - DNA, 17 bp, Artificial Sequence
  primer
agcattgtgttatcagc

SEQ ID NO: 32 - DNA, 17 bp, Artificial Sequence
  primer
gcagcattggtagcaac

SEQ ID NO: 33 - DNA, 22 bp, Artificial Sequence
  primer
gtgtggcaaggtggcaaagcca

SEQ ID NO: 34 - DNA, 25 bp, Artificial Sequence
  primer
ccgatctacaggcccaactcgatga